(19) United States
(12) Patent Application Publication
(10) Pub. No.: US 2024/0358619 A1
(43) Pub. Date: Oct. 31, 2024

(54) NAIL POLISH COLOR COAT COMPOSITION

(71) Applicant: L'OREAL, PARIS (FR)

(72) Inventors: Luke Zachary SHAPIRO, Matawan, NJ (US); Ramakrishnan HARIHARAN, Springfield, NJ (US); I-fan HSIEH, Scotch Plains, NJ (US)

(73) Assignee: L'OREAL, PARIS (FR)

(21) Appl. No.: 18/309,163

(22) Filed: Apr. 28, 2023

(51) Int. Cl. A61K 8/73; A61K 8/81; A61Q 3/02

(52) U.S. Cl. CPC A61K 8/731; A61K 8/8147; A61Q 3/02; A61K 2800/43

(57) ABSTRACT

A composition for use on the nails is provided, comprising a solvent system that includes at least one volatile, non-aqueous polar solvent; a water-insoluble nitrocellulose polymer; an acrylic copolymer with an acid value ranging from about 100 and about 170 mg KOH per gram; an epoxy resin; and a colorant. Methods including those for use as a color coat are also provided.

### FIELD OF THE INVENTION

The present invention relates to nail polish color coat compositions, and specifically to nail polish color coat compositions comprising certain acrylic polymers.

### BACKGROUND

Consumers use nail polish to cosmetically enhance the appearance of their nails or protect the nails from the abuses found in their everyday environment. Often this requires a nail polish that is formulated to provide a good shine.

Consumers also desire a durable nail polish. Lack of durability is often evidenced by unsightly chipping or peeling of the coating soon after the original coating has been applied, requiring at least in part a reapplication of the coating in an attempt to recreate the aesthetic appearance of the original nail coating. Durability is often a delicate balance, as coatings that are too soft can scuff and coatings that are too stiff suffer from chipping.

A continuing need exists to simultaneously provide both improved gloss and improved durability for nail polish color coats.

### BRIEF SUMMARY

The present invention is directed to a nail polish color coat composition (also referred to herein as “nail color coat composition,” or just “composition”). The nail polish color coat composition includes a solvent system. The solvent system includes at least one volatile, non-aqueous polar solvent. The nail polish color coat composition further includes a water-insoluble nitrocellulose polymer as well as an acrylic copolymer with an acid value ranging from about 100 and about 170 mg KOH per gram. The nail polish color coat composition further includes an epoxy resin and a colorant.

Advantageously, the acid number of the acrylic polymer may be between about 130 and about 170 mg KOH per gram, and/or be present in a concentration by weight in the nail color coat composition that is from about 0.5% to about 3% by weight in the color coat composition.

It may also be advantageous for the epoxy resin to be or include a toluenesulfonamide epoxy resin and/or be present in a concentration by weight in the nail color coat composition that is from about 8% to about 12%.

It may also be advantageous for a colorant to be or include a pigment and/or a pearl pigment and/or to be present in a concentration by weight in the nail color coat composition that is from about 0.1% to about 5%.

It may also be advantageous for the at least one volatile, non-aqueous polar solvent to include at least one volatile acetate, such as ethyl acetate, propyl acetate and butyl acetate, such as where these compounds are present in a concentration by weight such that a total concentration by weight of ethyl acetate, propyl acetate and butyl acetate in the nail color coat composition is from about 50% to about 90%.

It may also be advantageous for the nail color coat composition to further include sucrose acetate butyrate and/or acetyl tributyl citrate.

It may also be advantageous for the water-insoluble nitrocellulose polymer to be nitrocellulose such as to be present in a concentration by weight in the nail color coat composition that is from about 5% to about 15%.

According to certain embodiments, the nail polish color coat composition may meet a plurality or even all these above listed requirements.

According to another aspect of the invention, a method of treating a nail is provided. The method includes applying to the nail the above-described nail polish color coat composition.

### BRIEF DESCRIPTION OF THE DRAWINGS

Implementation of the present technology is described, by way of example only, with reference to the attached figure, wherein:

FIG. 1. Shows a graph of hardness over time for various compositions including a first acrylic polymer having a high acid number and one having a comparative acrylic polymer.

FIG. 2. Shows a graph of hardness over time for various compositions including a second acrylic polymer having a high acid number and one having a comparative acrylic polymer.

FIG. 3. Shows a graph of hardness over time for various compositions including a third acrylic polymer having a high acid number and one having a comparative acrylic polymer.

### DETAILED DESCRIPTION

As used herein, the expression “at least one” means one or more and thus includes individual components as well as mixtures/combinations.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. Unless otherwise indicated, all concentrations shown as percentages are concentrations by weight and also, unless otherwise indicated, relate to the entire nail color coat composition as a whole.

As used herein, articles such as “a” and “an” when used in a claim, are understood to mean one or more of what is claimed or described.

As used herein, the term “at least one” means one or more and thus includes individual components as well as mixtures/combinations.

As used herein, the terms “include”, “includes” and “including” are meant to be non-limiting.

“Film-former” or “film forming agent” or “film forming polymer” or “film forming resin” as used herein mean a polymer or resin that leaves a film on the substrate to which it is applied, for example, after a solvent accompanying the film-former has evaporated, absorbed into and/or dissipated on the substrate. A “silicone film-former” is a film-former that includes at least one silicone (a silicone atom bonded directly to an oxygen atom and also to a carbon atom such as in an organic moiety).

“Liquid” or “liquid cosmetic” or “liquid composition” means a composition having a fixed volume, flows to cover the bottom and assumes the shape of the portion of the container it fills and is slightly compressible (as disclosed in General chemistry, Fourth Edition 2005, p. 434.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Substantially free” as it is used herein means that while it is preferred that no amount of the specific component be present in the composition, it is possible to have small amounts of it in the compositions of the invention provided that these amounts do not materially affect at least one, preferably most, of the advantageous properties of the compositions of the invention. In certain embodiments, substantially free means less than about 2% of the identified ingredient, such as less than about 1%, such as less than about 0.5% based on the composition as a whole (or a particular component, if indicated). The term “anhydrous” means substantially free of water.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

The compositions and methods of the present invention can comprise, consist of, or consist essentially of the essential elements and limitations of the invention described herein, as well as any additional or optional ingredients, components, or limitations described herein or otherwise useful.

Nail color coat compositions of the present invention include a solvent system, a water-insoluble nitrocellulose polymer; an acrylic polymer; an epoxy resin; and a colorant. Each of these will be discussed in turn.

### Solvent System

Nail color coat compositions of the present invention include a solvent system. The function of the solvent system is to dissolve or disperse various components in the nail color coat composition and to evaporate at an appropriate rate in order to allow for the relatively fast formation of film that has desirable gloss and durability properties

The solvent system includes at least one volatile, non-aqueous polar solvent. The volatile, non-aqueous polar solvent is (1) “volatile” in that it evaporates at a rate at least as fast as butyl acetate; (2) is polar in that it includes at least one polar functional group (e.g., alcohol, ether, ester, ketone, hydroxyl, amine, amino, carboxyl, carbonyl, and the like), and (3) “non-aqueous” in that it is not water, and, in certain embodiments is not completely miscible in water throughout all concentrations at standard temperature and pressure.

According to certain embodiments, the at least one volatile, non-aqueous polar solvent includes an ester, such as one or more acetate compounds. In particularly notable embodiments, the acetate compound is selected from ethyl acetate, propyl acetate, butyl acetate, and combinations thereof. In certain embodiments the one or more acetate compounds are used as a majority portion of the at least one volatile, non-aqueous polar solvent, meaning that the proportion by weight of the one or more acetate compounds in the total amount at least one volatile, non-aqueous polar solvent is more than 50%, such as more than about 75%, such as more than about 90%.

According to certain embodiments, the at least one volatile, non-aqueous polar solvent is present in the nail color coat composition in a concentration by weight that is from about 50% or 60% to about 75%, 80% or 90%. In notable embodiments, the at least one volatile, non-aqueous polar solvent comprises, has a majority fraction (and more notably at least about 90% of the total volatile, non-aqueous polar solvents) that is a mixture of volatile acetates such as ethyl acetate, propyl acetate and butyl acetate. In other notable embodiments, the nail color coat composition has a total concentration by weight of ethyl acetate, propyl acetate and butyl acetate in the nail color coat composition is from about 50% to about 90%.

According to certain other embodiments the at least one volatile, non-aqueous polar solvent includes one or more monoalcohols, such as C2-C5 monoalcohol such as ethanol or isopropanol. In certain embodiments the one or more monoalcohols are used as a minority portion of the at least one volatile, non-aqueous polar solvent, meaning that the proportion by weight of the one or more monoalcohols in the total amount at least one volatile, non-aqueous polar solvent is less than 50%, such as less than about 25%, such as less than about 10%.

### Water-Insoluble Nitrocellulose Polymer

Nail color coat compositions of the present invention include a water-insoluble nitrocellulose polymer (e.g., cellulose nitrate). The nitrocellulose polymer may be formed by nitrating cellulose by exposure to acids such as nitric acid and/or sulfuric acid. Desirably the nitrocellulose is soluble in the solvent system and more particularly soluble in the one or more volatile, non-aqueous polar solvents. The water-insoluble nitrocellulose polymer serves as a film-former in the nail color coat composition. The nitrocellulose may be present in a concentration by weight in the nail color coat composition that is from about 5%, 6% or 7% to about 10%, 12% or 15% by weight.

Acrylic Polymer Having Acid Number from about 100 mg KOH Per Gram to about 170 mg KOH Per Gram

Nail color coat compositions of the present invention include an acrylic (co) polymer having an acid number (also known as acid value) that ranges from about 100 mg KOH per gram and about 170 mg KOH per gram. Acid value or acid number is reflective of the relative amount of carboxyl groups in the acrylic (co) polymer and is generally measured using titration, as known to one skilled in the art. The acrylic (co) polymer having an acid number will be formed from one or more types of ethylenically unsaturated monomeric substituents.

According to certain embodiments, the acid number of the acrylic polymer may be from about 130 to about 170 mg KOH per gram

According to certain other embodiments, the acid number of the acrylic polymer is from about 130, 135, 140, 145, or 150 mg KOH per gram to about 150 or 170 mg KOH per gram.

The present inventors have found that use of these relatively high acid value acrylates provide a combination of beneficial properties, particularly when used in combination with other formulation components detailed herein.

The acrylic (co) polymer having an acid number that ranges from about 130 and about 170 mg KOH per gram may have an INCI designation of, for example, Acrylates Copolymer.

In certain embodiments, the acrylic polymer having an acid number that ranges from about 130 and about 170 mg KOH per gram may have a softening point, as measured by the Ball & Ring method, known to those skilled in the art, that ranges from about 110° C. to about 125° C.

Suitable acrylic polymers having acid number that ranges from about 130 mg KOH per gram and about 170 mg KOH per gram include those available from Estron Chemical, Inc of Calvert City, KY and sold under the trade names EPOMATT G-151, EPOMATT G-152, EPOMATT G-154 or ISOCRYL G-265.

The acrylic copolymer with an acid value ranging from about 130 and about 170 mg KOH per gram is present in a concentration by weight in the nail color coat composition that is from less than about 3% by weight, such as from about 0.2%, 0.5% or 1% to about 2%, 2.5% or 3% by weight.

### Epoxy Resin

Nail color coat compositions of the present invention include an epoxy resin. In some embodiments, the epoxy resin has a glass transition temperature (Tg) below about 100° C., preferably below about 80° C. Examples of suitable epoxy resin include those soluble in butyl acetate. According to certain embodiments the epoxy resin is tosylamide epoxy resin, for example a toluenesulfonamide of the condensation product of 4,4′-Isopropylidenediphenol/Epichlorohydrin Copolymer. Suitable examples include those having an INCI designation of Tosylamide/Epoxy resin.

Non-limiting examples of suitable epoxy resins include Tosylamide/Epoxy resin, such as those sold by Estron Chemical under the trade name Polytex, eg E-75, E-100, NX-55, NX-3214, preferably Polytex NX-55.

The epoxy resin may be present in a concentration by weight in the nail color coat composition that is from about 2%, or 5% to about 10% 12%, or 15%.

According to certain embodiments, the acrylic copolymer with an acid value ranging from about 100 and about 170 mg KOH per gram and the epoxy resin be present in a ratio of concentration by weight of acrylic copolymer with an acid value ranging from about 100 and about 170 mg KOH per gram to concentration by weight of epoxy resin that is in a range from about 0.02:1, 0.03:1, 0.05:1, 0.1:1 to about 0.2:1, 0.4:1, 0.6:1, 0.8:1.

### Other Ingredients

Nail color coat compositions of the present invention may include various other ingredients without significantly compromising the beneficial properties.

For example, according to certain embodiments, plasticizers other than the epoxy resin may be included in the nail color coat composition. Examples of such other plasticizers include polar molecules having multiple ester groups such as those having a molecular weight in a range from about 250 or 400 to about 1000 or 1500. Examples include sucrose acetate butyrate and acetyl tributyl citrate. In certain embodiments the nail color coat composition includes an ingredient selected from a group consisting of sucrose acetate butyrate and acetyl tributyl citrate. Sucrose acetate butyrate is sold under SAIB 90 or SUSTANE SAIB by Eastman Chemical of Kingsport, TN. Acetyl tributyl citrate is sold under Citroflex brand by Vertellus, or the Uniplex brand by Unitex Chemical of Greensboro, North Carolina.

Other exemplary plasticizers include dipropylene glycol dibenzoate, and 1,2-propylene glycol dibenzoate, trioctyl citrate, triethyl citrate, acetyl tributyl citrate, or tributyl citrate.

These other plasticizers may be present in concentrations ranging from about 1%, 2% or 3% to about 10%, 15% or 25% by weight.

As another example, according to certain embodiments, additional film forming polymers or resins may be included in the nail color coat composition. The film-forming polymer can comprise polyester resins formed by reaction of a polyhydric alcohol with a basic polyacid, for example phthalic acid, such as the commercial product sold by Unitex Chemical Corporation under the name UNIPLEX 670-P, which is a polyester resin obtained by reaction of trimellitic acid, neopentyl glycol and adipic acid. (Meth)acrylic resins, according to the disclosure, can include copolymers of methyl methacrylate with butyl acrylate, butyl methacrylate, isobutyl methacrylate or isobornyl methacrylate, for example the commercial products PARALOID DM-55, PARALOID B48N, PARALOID B66 and ELVACITE 2550; copolymers of isobutyl methacrylate and butyl methacrylate, for example, the commercial product ELVACITE 2046; and isobutyl methacrylate polymers, for example, PARALOID B67.

Another example of a suitable film forming polymer is acrylic dispersion in isododecane, containing in total 10% acrylic acid, 10% methyl acrylate, 60% ethyl acrylate and 20% isobornyl acrylate, such as the product with the INCI name: ACRYLATES/ISOBORNYL ACRYLATE COPOLYMER sold under the trade name MEXOMERE PBM® from the company CHIMEX.

Another example of a suitable resin is a ketone-aldehyde resin, e.g., a copolymer comprising an aromatic ketone. Potential ketone-aldehyde resins may include, but are not limited to, those described in U.S. Pub. No. 2009/0012245, U.S. Pat. Nos. 7,183,372 and 7,101,958, or those sold by Evonik Industries under the TEGO® Variplus brand, such as TEGO® Variplus SK.

As another example, according to certain embodiments, mattifying agents may also be included in the nail color coat composition. Examples include silica, aluminosilicates, borosilicates, and the like. These mattifying agents may be present in concentrations ranging from about 0.25%, 0.5% or 1% to about 1%, 2% or 5% by weight.

As another example, according to certain embodiments, hydrocarbon oils may also be included in the nail color coat composition. “Hydrocarbon oil” refers to a compound which is liquid at room temperature, is insoluble in water and is of hydrophobic character. The hydrocarbon oils used in conformity with the invention are typically synthetic oils, mineral, vegetable or animal oils, unsaturated fatty alcohols, and esters of fatty acids and lower C₂-C₄ mono- or polyalcohols.

The one or more hydrocarbon oils may be present in an amount ranging from about 1% to about 20% by weight, more preferably from about 1% to about 10% by weight, and still more preferably from about 0.5% to about 5% by weight.

As another example, according to certain embodiments, non-polar silicone copolymers may be included in the nail color coat composition. In certain embodiments, the non-polar silicone copolymer may comprise repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the product sold under the tradename KP-543 or KP-545, or acrylates/stearyl acrylate/dimethicone acrylates copolymers, such as those commercially available from Shin-Etsu, for example, the product sold under the tradename KP-561, and acrylates/behenyl acrylate/dimethicone acrylates copolymer, such as those commercially available from Shin-Etsu, for example, the product sold under the tradename KP-562.

The non-polar silicone copolymer may present in an amount ranging from about 0.01% to about 2% by weight. Preferably, the non-polar silicone copolymer may present in an amount ranging from about 0.01% to about 1% by weight, more preferably from about 0.05% to about 0.6% by weight.

In certain notable embodiments, the nail polish color coat composition may include certain components such as water and/or colorants (coloring agent). However, in other embodiments, it is generally preferred that the nail color coat composition be substantially free of water and/or colorants.

The commonly used colorants that the nail color coat composition may include dyes, pigments, pearls & effect pigments.

Dyes include, for example, fat-soluble dyes such as Sudan red, DC Red 17, DC Green 6, β-carotene, Sudan brown, DC Yellow 11, DC Violet 2, DC Orange 5 and quinoline yellow.

Pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7. D&C Red No. 5 and 6, and D&D Red No. 34, as well as lakes such as D&C Yellow Lake No. 5 and D&C Red Lake No. 2.

Pearlescent pigments include, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Nail color coat compositions of the present invention may be made by methods known in the art, such as by optionally premixing certain ingredients into the one more solvents and blending various ingredients together in careful manner to provide a homogeneous mixture.

### Methods of Use

Nail color coat compositions may be applied onto a nail such as human fingernail or toenail to form a color coat, which is a layer of nail polish. According to certain embodiments a color coat composition is preferably applied contiguously to a mammalian nail with or without, more preferably with, one or more succeeding layers applied to the resulting color coat. According to certain embodiments the nail color coat formed thereon by commonly used methods such as brushing or spraying. A suitable topcoat composition, such as one substantially free of colorants may be applied over the color coat composition. The topcoat composition may include an acrylic copolymer with an acid value ranging from about 145 and about 170 mg KOH per gram and optionally a cellulose ester of C2-C4 organic acid (such as cellulose acetate butyrate). The acrylic copolymer with an acid value ranging from about 145 and about 170 mg KOH per gram and the optional C2-C4 organic acid (such as cellulose acetate butyrate) may be present in concentrations by weight in the topcoat of about 0.5% to 15% and 1% to 3% respectively. Otherwise, the topcoat may include other commonly used topcoat ingredients such as nitrocellulose and other ingredients disclosed herein with reference to the color coat components. Such color coats and topcoats may be packaged together such as in a kit.

The following examples are intended to illustrate the invention without limiting the scope as a result. The percentages are given on a weight basis.

### EXAMPLES

### Example 1: Flexibility

Compositions were prepared to evaluate the flexibility of films formed from four polymeric materials. Specifically, the following materials were evaluated:
(1) an acrylic copolymer having an acid value between 130 mg KOH per gram and 150 mg KOH per gram [Example E1, consistent with embodiments of the invention];
(2) an acrylic copolymer having an acid value between 145 mg KOH per gram and 165 mg KOH per gram [Example E2, consistent with embodiments of the invention];
(3) an acrylic copolymer having an acid value between 160 mg KOH per gram and 170 mg KOH per gram [Example E3, consistent with embodiments of the invention];
(4) an acrylic copolymer having an acid value of 0 mg KOH per gram [Example C1].

A test solution of each of the materials above was prepared by dissolving various materials to a concentration by weight in ethyl acetate of 40% (except for Example C1, which was 50% active). The test solutions were evaluated using a conical mandrel test, ASTM test D522-93a (2008), “Standard Test Methods for Mandrel Bend Test of Attached Organic Coatings.”

Cold-rolled steel test strips are used as substrates (22 gauge, 0.8 mm thick), preferably 100 mm×150 mm long/wide. The strips are cleaned such as with volatile solvent, to remove residual oil. Uniform coatings are applied to the test strips and allow them to air dry. The coated dried samples are conditioned for at least about 2 hrs. and 50% relative humidity. A conical mandrel tester having a smooth steel 200 ml long cone with a diameter of 3 mm at one end and 38 mm at the other end is employed.

With the operating lever of the apparatus in a horizontal position, the test specimen is slipped between the mandrel and the drawbar with the finish side towards the drawbar. The specimen is rigidly clamped in a vertical position adjacent to the mandrel by placing the long edge behind the clamping bar in such a manner that the panel is always set up to the narrow end of the mandrel.

The lever is moved through about 180° at uniform velocity to bend the specimen approximately 135°.

To determine crack resistance under more simulated use conditions, the bend time should be about 1 s. The bent surface of the specimen is examined immediately with the unaided eye for cracking.

The results of the test indicated that; Example E2 and Example E3 showed minimal cracking after day 1. Example E1 showed some cracking after day 1; Example C1 showed some cracking and additionally showed significant buckling after day 1.

### Example 2: Hardness for Nail Color Coat Compositions

Compositions were prepared to evaluate the hardness of color coat compositions. Specifically, the following color coat compositions were evaluated:
(1) composition comprising different levels of an acrylic copolymer having an acid value between 130 mg KOH per gram and 150 mg KOH per gram and epoxy resin [Example E7.1, E7.2, E7.3, E7.4, E7.5 consistent with embodiments of the invention];
(2) composition comprising different levels of an acrylic copolymer having an acid value between 145 mg KOH per gram and 165 mg KOH per gram and epoxy resin [Example E8.1, E8.2, E8.3, E8.4, E8.5, consistent with embodiments of the invention];
(3) composition comprising different levels of an acrylic copolymer having an acid value between 160 mg KOH per gram and 170 mg KOH per gram and epoxy resin [Example E9.1, E9.2, E9.3, E9.4, E9.5, consistent with embodiments of the invention].
(4) composition comprising an acrylic copolymer having an acid value 0 mg KOH per gram and epoxy resin. [Example C2]

### Sample Preparation

Samples were prepared by combining solvents, nitrocellulose, acrylic polymer, epoxy resin and other plasticizers, other film-forming polymers and other ingredients in a jar. The blend was allowed to let sit 12 to 24 hours. It was then mixed for three minutes with overhead mixer until uniform. The samples were then allowed to sit for 24 hours before testing so any air bubbles would dissipate. The test nail color coat compositions were evaluated by measuring hardness at room temperature.

### Test Description

To perform the test, a 5×4-inch glass plate and a 6 mil Bird applicator are thoroughly cleaned with acetone and wiped KLEENEX tissue until dry. The glass plate is placed in the center of an automatic draw down machine of a Erichsen Dry Time Apparatus. The speed of the drawdown machine is set for 25 mm/sec. The Bird Applicator is set on the glass plate and flush against the moving bar. Three ml of test composition is placed across the width of the glass plate. The glass place is held, and the start button is pressed on the drawdown machine to form a homogeneous film across the length of the plate without moving the position on the plate. The film is drawn down using the 6 mil Bird Applicator. The drawdown bar is reset, and residue is cleaned from glass plate and drawdown machine with acetone. The film is allowed to dry about 24 hours (1 day) and initial hardness reading is taken. Additional measurements are taken at 5, 7, 10, and 14 days after initial draw down.

The various nail color coat compositions are shown below in Table 1, Table 2 & Table 3. These nail color coat compositions were evaluated for hardness, adhesion, and gloss.

TABLE 1

| | EXAMPLE | | | | | |
|---|---|---|---|---|---|---|
| COMPONENT | E7.1 | E7.2 | E7.3 | E7.4 | E7.5 | C2 |
| Acrylic copolymer, | 1% | 3% | 5% | 7.5% | 10% | — |
| acid value 130-150 | | | | | | |
| (mg KOH per gram) | | | | | | |
| Acrylic copolymer, | — | — | — | — | — | 1.1% |
| acid value 0 (mg | | | | | | |
| KOH per gram) | | | | | | |
| Epoxy resin | 7.9% | 7.9% | 7.9% | 7.9% | 7.9% | 7.9% |
| Nitrocellulose | 9.8% | 9.5% | 9.3% | 9.0% | 8.8% | 9.6% |
| Solvents, including | 73% | 71% | 69% | 67% | 65% | 73% |
| acetates | | | | | | |
| Pigments | 1% | 1% | 1% | 1% | 1% | 1% |
| Other components | qs | qs | qs | qs | qs | qs |
| Total | 100% | 100% | 100% | 100% | 100% | 100% |

*Apparent errors due to rounding

TABLE 2

| | EXAMPLE | | | | | |
|---|---|---|---|---|---|---|
| COMPONENT | E8.1 | E8.2 | E8.3 | E8.4 | E8.5 | C2 |
| Acrylic copolymer, | 1% | 3% | 5% | 7.5% | 10% | — |
| acid value 145-165 | | | | | | |
| (mg KOH per gram) | | | | | | |
| Acrylic copolymer, | — | — | — | — | — | 1.1% |
| acid value 0 (mg | | | | | | |
| KOH per gram) | | | | | | |
| Epoxy resin | 7.9% | 7.9% | 7.9% | 7.9% | 7.9% | 7.9% |
| Nitrocellulose | 9.8% | 9.5% | 9.3% | 9.0% | 8.8% | 9.6% |
| Solvents, including | 73% | 71% | 69% | 67% | 65% | 73% |
| acetates | | | | | | |
| Pigments | 1% | 1% | 1% | 1% | 1% | 1% |
| Other components | qs | qs | qs | qs | qs | qs |
| Total | 100% | 100% | 100% | 100% | 100% | 100% |

*Apparent errors due to rounding

TABLE 3

| | EXAMPLE | | | | | |
|---|---|---|---|---|---|---|
| COMPONENT | E9.1 | E9.2 | E9.3 | E9.4 | E9.5 | C2 |
| Acrylic copolymer, | 1% | 3% | 5% | 7.5% | 10% | — |
| acid value 160-170 | | | | | | |
| (mg KOH per gram) | | | | | | |
| Acrylic copolymer, | — | — | — | — | — | 1.1% |
| acid value 0 (mg | | | | | | |
| KOH per gram) | | | | | | |
| Epoxy resin | 7.9% | 7.9% | 7.9% | 7.9% | 7.9% | 7.9% |
| Nitrocellulose | 9.8% | 9.5% | 9.3% | 9.0% | 8.8% | 9.6% |
| Solvents, including | 73% | 71% | 69% | 67% | 65% | 73% |
| acetates | | | | | | |
| Pigments | 1% | 1% | 1% | 1% | 1% | 1% |
| Other components | qs | qs | qs | qs | qs | qs |
| Total | 100% | 100% | 100% | 100% | 100% | 100% |

*Apparent errors due to rounding

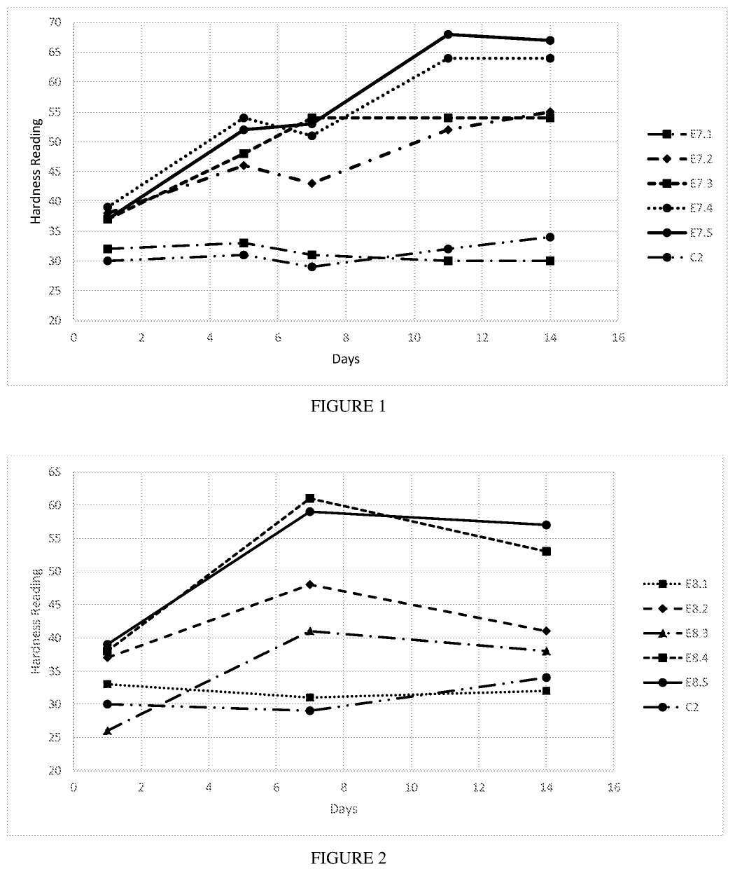

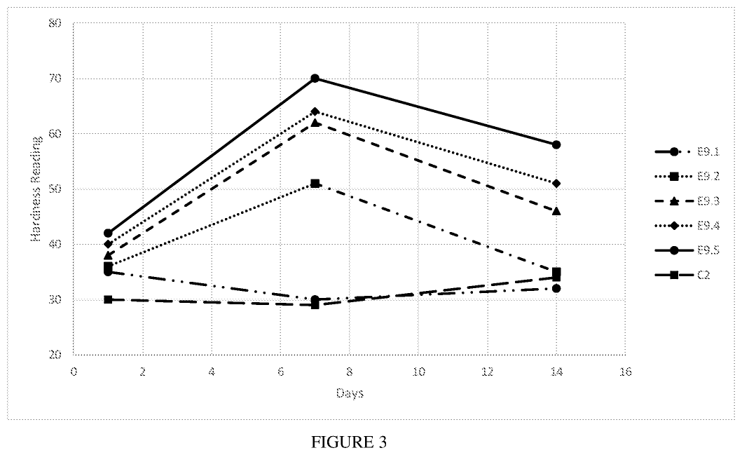

The various nail color coat composition hardness results are shown in FIG. 1, FIG. 2 & FIG. 3. The results indicate that color coat compositions with lower level of (E7.1) an acrylic copolymer with an acid value ranging from about 130 and about 150 mg KOH per gram and epoxy resin; lower level of (E8.1) an acrylic copolymer with an acid value ranging from about 145 and about 165 mg KOH per gram and epoxy resin; and lower level of (E9.1) an acrylic copolymer with an acid value ranging from about 160 and about 170 mg KOH per gram and an epoxy resin all shows excellent hardness stability.

### Example 3: Adhesion for Nail Color Coat Compositions

Compositions were prepared to evaluate the adhesion of color coat compositions. Specifically, the following color coat compositions were evaluated:
(1) composition comprising different levels of an acrylic copolymer having an acid value between 130 mg KOH per gram and 150 mg KOH per gram and epoxy resin [Example E7.1, E7.2, E7.5, described above];
(2) composition comprising different levels of an acrylic copolymer having an acid value between 145 mg KOH per gram and 165 mg KOH per gram and epoxy resin [Example E8.1, E8.2, E8.5, described above];
(3) composition comprising different levels of an acrylic copolymer having an acid value between 160 mg KOH per gram and 170 mg KOH per gram and epoxy resin [Example E9.1, E9.2, E9.5, described above].

### Sample Preparation

Samples were prepared by combining solvents, nitrocellulose, acrylic polymer, epoxy resin and other plasticizers, other film-forming polymers and other ingredients in a jar. The blend was allowed to let sit 12 to 24 hours. It was then mixed for three minutes with overhead mixer until uniform. The samples were then allowed to sit for 24 hours before testing. The nail polish film is drawn down using the 6-mil bird applicator on vitro nail substrate. The vitro nail samples were then allowed to sit for 24 hours before testing. The test nail color coat compositions were evaluated by measuring adhesion.

### Test Description

To perform the test, a vitro nail substrate and a 6-mil bird applicator are thoroughly cleaned with acetone and wiped KLEENEX tissue until dry. The nail polish film is drawn down using the 6-mil bird applicator on vitro nail substrate. The samples were then allowed to sit for 24 hours before testing. The test samples were evaluated using ASTM D3359 “Standard test method for measuring adhesion by tape test”. A cross hatch tool is used to scrape the film on the vitro nail. The substrate is then rotated 90 degrees and scraped again. A piece of tape is applied on top of the scraped area and lifted. Amount of product remaining is evaluated for adhesion.

Adhesion results for various nail color coat compositions are shown in Table 4.

TABLE 4

| | Example | | |
|---|---|---|---|
| Adhesion | E7.1: Rating 3 | E8.1: Rating 1 | E9.1: Rating 2 |
| results | E7.2: Rating 3.5 | E8.2: Rating 4 | E9.2: Rating 4 |
| | E7.5: Rating 5 | E8.5: Rating 5 | E9.5: Rating 5 |

Rating 1: Detachment of small flakes, less than 5% affected
Rating 2: Coating is flaked along the edges or in long strips, between 5% and 15% affected
Rating 3: Coating is flaked along the edges in long strips or wholly in large ribbons with parts of squares being removed, between 15% and 35% affected
Rating 4: Coating has flaked along the edges of cuts in flakes or large strips with squares that have detached wholly or partly. 35% to 65% affected
Rating 5: Any flaking beyond a 4 rating

The results indicate that color coat compositions with lower level of (E7.1) an acrylic copolymer with an acid value ranging from about 130 and about 150 mg KOH per gram and epoxy resin; lower level of (E8.1) an acrylic copolymer with an acid value ranging from about 145 and about 165 mg KOH per gram and epoxy resin; and lower level of (E9.1) an acrylic copolymer with an acid value ranging from about 160 and about 170 mg KOH per gram and an epoxy resin all shows excellent adhesion.

### Example 4: Gloss/Shine for Nail Color Coat Compositions

Compositions were prepared to evaluate the shine or gloss of color coat compositions. Specifically, the following color coat compositions were evaluated:
(1) composition comprising an acrylic copolymer having an acid value between 130 mg KOH per gram and 150 mg KOH per gram and epoxy resin [Example E7.1, described above];
(2) composition comprising different levels of an acrylic copolymer having an acid value between 145 mg KOH per gram and 165 mg KOH per gram and epoxy resin [Example E8.1, described above];
(3) composition comprising different levels of an acrylic copolymer having an acid value between 160 mg KOH per gram and 170 mg KOH per gram and epoxy resin [Example E9.1, described above].
(3) composition comprising an acrylic copolymer having an acid value 0 mg KOH per gram and epoxy resin. [Example C2, described above]

### Sample Preparation

Samples were prepared by combining solvents, nitrocellulose, acrylic polymer, epoxy resin and other plasticizers, other film-forming polymers and other ingredients in a jar. The blend was allowed to let sit 12 to 24 hours. It was then mixed for three minutes with overhead mixer until uniform. The samples were then allowed to sit for 24 hours before testing so any air bubbles would dissipate. The test nail color coat compositions were evaluated by measuring gloss at room temperature.

### Test Description

To measure gloss, a black and white Laneta card is placed onto a vacuum plate. A film is drawn down using a 6 mil Bird Applicator onto the card and allowed to dry two hours under ambient conditions. A Byk-Gardener Micro-Tri-Glossmeter is powered in its holder and allowed to run autodiagnosis and calibration. If requested, the standard glass plate is cleaned using a glass cleaning cloth and calibration is started by pressing the mode wheel. Readings at room temperature are taken by pressing the start button.

Gloss results for various nail color coat compositions are shown in Table 5.

TABLE 5

Example

| | E7.1 | E8.1 | E9.1 | C2 |
|---|---|---|---|---|
| Gloss | 66.8 | 68.1 | 70 | 60.3 |
| results | | | | |

The results indicate surprisingly good gloss performance for the following composition combinations, (E7.1) an acrylic copolymer with an acid value ranging from about 130 and about 150 mg KOH per gram; and an epoxy resin; (E8.1) an acrylic copolymer with an acid value ranging from about 145 and about 165 mg KOH per gram; and an epoxy resin; (E9.1) an acrylic copolymer with an acid value ranging from about 160 and about 170 mg KOH per gram; and an epoxy resin as compared to composition comprising an acrylic copolymer having an acid value 0 mg KOH per gram.

What is claimed:

1. A composition for use on nails, comprising:
a solvent system comprising at least one volatile, non-aqueous polar solvent;
a water-insoluble nitrocellulose polymer;
an acrylic copolymer with an acid value ranging from about 100 and about 170 mg KOH per gram;
an epoxy resin; and
a colorant.

2. The composition of claim 1, wherein the acrylic copolymer has an acid value between about 130 and about 170 mg KOH per gram.

3. The composition of claim 1, wherein the acrylic copolymer has an acid value between about 160 and about 170 mg KOH per gram.

4. The composition of claim 1, wherein the colorant comprises pigments, pearl pigments, or combinations thereof.

5. The composition of claim 1, wherein the at least one volatile, non-aqueous polar solvent comprises at least one volatile acetate.

6. The composition of claim 1, wherein the epoxy resin is a Tosylamide/Epoxy resin.

7. The composition of claim 1, further comprising an ingredient selected from a group consisting of sucrose acetate butyrate and acetyl tributyl citrate.

8. The composition of claim 1, wherein the solvent system comprising at least one volatile, non-aqueous polar solvent comprises ethyl acetate and butyl acetate each in a respective concentration by weight such that a total concentration by weight of ethyl acetate and butyl acetate in the composition is from about 50% to about 90%.

9. The composition of claim 1, wherein the water-insoluble nitrocellulose polymer is nitrocellulose and wherein the nitrocellulose is present in a concentration by weight in the composition that is from about 5% to about 15%.

10. The composition of claim 1, wherein the acrylic copolymer with an acid value ranging from about 100 and about 170 mg KOH per gram is present in a concentration by weight in the composition that is from about 0.5% to about 3% by weight.

11. The composition of claim 1, wherein the acrylic copolymer with an acid value ranging from about 100 and about 170 mg KOH per gram is present in a concentration by weight in the composition that is from about 0.5% to about 2% by weight.

12. The composition of claim 1, wherein the epoxy resin is Tosylamide/Epoxy resin, and the Tosylamide/Epoxy resin is present in a concentration by weight in the composition that is from about 5% to about 10%.

13. The composition of claim 1, wherein the solvent system comprising at least one volatile, non-aqueous polar solvent comprises ethyl acetate, propyl acetate and butyl acetate each in a respective concentration by weight such that a total concentration by weight of ethyl acetate, propyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein the acrylic copolymer with an acid value ranging from about 130 and about 170 mg KOH per gram is present in a concentration by weight in the composition that is from about 0.5% to about 3% by weight, and wherein the epoxy resin is Tosylamide/Epoxy resin and the Tosylamide/Epoxy resin is present in a concentration by weight in the composition that is from about 5% to about 10%, wherein the colorant is present in concentration by weight in the composition that is from about 0.1 to about 10%.

14. The composition of claim 1, further comprises additional film forming polymer, an additive, a silicone, a filler, a UV absorbing agent or a combination thereof.

15. A method of treating a nail, comprising:
applying to the nail a composition comprising:
a solvent system comprising at least one volatile, non-aqueous polar solvent;
a water-insoluble nitrocellulose cellulose polymer;
an acrylic copolymer with an acid value ranging from about 100 and about 170 mg KOH per gram;
an epoxy resin; and
a colorant.

16. The method of claim 15, wherein the acrylic copolymer has an acid value between about 160 and about 170 mg KOH per gram.

17. The method of claim 15, wherein the at least one volatile, non-aqueous polar solvent comprises at least one volatile acetate.

18. The method of claim 15, wherein the epoxy resin is a Tosylamide/Epoxy resin.

19. The method of claim 15, wherein the acrylic copolymer with an acid value ranging from about 130 and about 170 mg KOH per gram is present in a concentration by weight in the composition that is from about 0.5% to about 15% by weight.

20. The method of claim 15, wherein the solvent system comprising at least one non-aqueous polar solvent comprises ethyl acetate and butyl acetate each in a respective concentration by weight such that a total concentration by weight of ethyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein the acrylic copolymer with an acid value ranging from about 130 and about 170 mg KOH per gram is present in a concentration by weight in the composition that is from about 0.5% to about 3% by weight, and wherein the epoxy resin is a Tosylamide/Epoxy resin and the is a Tosylamide/Epoxy resin is present in a concentration by weight in the composition that is from about 5% to about 10%, wherein the colorant is present in concentration by weight in the composition that is from about 0.1 to about 10%.
